(19) United States
(12) Patent Application Publication
(10) Pub. No.: US 2019/0183797 A1
(43) Pub. Date: Jun. 20, 2019

(54) BETULIN-CONTAINING WATER-IN-OIL FOAMS AND COMPOSITIONS THEREOF

(71) Applicants: Rolf DANIELS, Tübingen (DE); Tobias ZAHN, Dublin (IE); Amryt Research Limited, Dublin (IE)

(72) Inventors: Rolf Daniels, Tubingen (DE); Tobias Zahn, Dublin (IE)

(21) Appl. No.: 16/318,805

(22) PCT Filed: Jul. 18, 2017
(86) PCT No.: PCT/US17/42610

§ 371 Date: Jan. 18, 2019

Related U.S. Application Data
(60) Provisional application No. 62/363,634, filed on Jul. 18, 2016.

(51) Int. Cl. A61K 9/12; A61K 9/107; A61K 36/185; A61K 47/14; A61K 47/44; A61P 17/02

(52) U.S. Cl. CPC A61K 9/122; A61K 9/107; A61P 17/02; A61K 47/14; A61K 47/44; A61K 36/185

(57) ABSTRACT

The present disclosure relates to foams comprising outer birch bard extracts. Stable compositions thereof, as well as methods of producing such foams, and methods of using of such foams are also described herein. The foams of the present disclosure contain triterpenes, which are known to improve wound healing.

### PRIORITY INFORMATION

This application claims the benefit of U.S. Provisional Patent Application No. 62/361634, filed on Jul. 18, 2016 and entitled “Ex Vivo Skin Permeation of Betulin from Water-in-Oil Foams,” the disclosure of which is hereby incorporated by reference in its entirety for all purposes.

### FIELD OF THE DISCLOSURE

The present disclosure relates to pharmaceutical formulations derived from the extracts of birch bark.

### BACKGROUND OF THE DISCLOSURE

The triterpenes found in birch bark extracts are known to have wound healing properties. Methods of extracting these triterpenes from birch bark are reported in U.S. Pat. No. 7,482,383. These methods provide solid birch bark extracts that may be used in pharmaceutical formulations. For example, emulsions containing such extracts are described in U.S. Pat. No. 7,482,383, and oleogels containing such extracts are described in U.S. Pat. Nos. 9,352,041; 8,828,444 and 8,536,380.

For clinical use in wound healing, an oleogel or cream must be applied by touch (e.g., by application with the fingers, a spatula or other applicator) to the area of the skin where treatment is needed. Touch application is disadvantageous for the treatment of certain skin conditions (e.g., epidermolysis bullosa) because the simple act of applying the oleogel or cream may lead to worsening of the skin condition. Touch application to injured skin can also cause significant physical stress and can be painful to the patient. As a result, patient compliance is at risk, which is particularly problematic for the treatment of chronic wounds.

Therefore, a need remains for wound-healing formulations containing solid birch bark extracts that may be applied to the skin without worsening the patient's condition or causing additional pain.

The present disclosure provides clinically-advantageous wound-healing formulations with improved rheological properties which overcome the disadvantages of the known solid birch bark-containing emulsions and oleogels.

### SUMMARY OF THE DISCLOSURE

The present disclosure provides emulsion foams comprising solid birch bark extracts dispersed in one or more nonpolar liquids. The solid birch bark extracts described herein may be formulated as emulsion foams that possess clinically-advantageous rheological properties.

In various embodiments of the present disclosure, the foams as described herein are prepared from emulsions of solid birch bark extracts that contain at least about 70% by weight of betulin and one or more triterpenes selected from the group consisting of betulinic acid, oleanolic acid, erythrodiol and lupeol.

The solid birch bark extracts can be dispersed in a nonpolar solvent. In some embodiments, the nonpolar liquid is selected from the group consisting of sunflower oil, medium chain triglycerides, and paraffin. In some embodiments, the nonpolar liquid comprises at least one triglyceride. In a specific embodiment, the triglyceride is medium chain triglycerides. In other embodiments, the nonpolar liquid comprises at least one C7 or greater hydrocarbon. In still other embodiments, the nonpolar liquid comprises one or more vegetable oils. In specific embodiments, the nonpolar liquid comprises sunflower oil.

Nonpolar liquids with units of unsaturation such as oils and other lipids can undergo autoxidation. Measuring the peroxide value is a standard method used to determine the extent to which this process occurs. Higher peroxide values equate to more rancidity on the quantitative scale. In one embodiment, the present disclosure provides emulsion foams, wherein the nonpolar liquid has a peroxide value less than about 10. In another embodiment, the peroxide number is no more than about 3. In still other embodiments, the peroxide value is less than 15, less than 14, less than 13, less than 12, less than 11, less than 10, less than 9, less than 8, less than 7, less than 6, less than 5, less than 4, less than 3, less than 2, less than 1, and including all values therebetween.

The foams of the present disclosure are prepared from emulsions. In some embodiments, the foams comprising solid birch bark extracts dispersed in one or more nonpolar liquids further comprise water. Usually foams are based on oil-in-water emulsions where the propellant (commonly propane/butane mixtures) is mixed with the dispersed lipid phase of the emulsion. In contrast, various embodiments of the present disclosure describe foams, wherein the foams comprise water-in-oil emulsions. This concept allows for the combination of the advantages of a touchless application with those of the healing effects of the birch bark triterpenes in a formulation which advantageously contains only triterpene extracts (TE), oil and water.

The present disclosure provides a foam comprising an emulsion, referred to herein interchangeably as an emulsion foam. The present disclosure provides a foam comprising an emulsion, wherein the emulsion comprises the oleogel provided herein. Thus, in various other embodiments, the emulsion used to prepare the foam is prepared from an oleogel comprising solid birch bark extracts. Mixed with oils, the birch bark extract of the present disclosure forms stable oleogels, which can absorb up to 60% of water forming a water-in-oil emulsion.

In one embodiment, the foam of the present disclosure provided by an emulsified oleogel, wherein the oleogel comprises about 5 wt. % to about 10 wt. % solid birch bark extract and the emulsion is a water-in-oil emulsion consisting of the oleogel and about 20 wt. % to about 30 wt. % of water. In another embodiment, the oleogel being emulsified comprises about 7 wt. % solid birch bark extract and the amount of water in the emulsion is about 25 wt. %.

In another embodiment, the present disclosure provides an emulsion foam comprising about 1% to about 20% by weight of particles of the solid birch bark extract dispersed in about 80% to about 99% of one or more nonpolar liquids, wherein the solid birch bark extracts are dispersed in a suitable nonpolar liquid to form an oleogel, wherein the oleogel is emulsified, and wherein the emulsion is used to prepare a foam comprising the solid birch bark extract.

In other embodiments, the present disclosure provides a foam comprising an emulsion, wherein the emulsion does not comprise, or is not prepared from an oleogel. In some embodiments, the foam is a water-in-oil foam. In other embodiments, the foam is an oil-in-water foam.

In various other embodiments, the emulsion foams of the present disclosure are essentially free of viable micro-organisms, fulfilling the requirements of sterile products according to pharmacopeias, e.g. USP, or PhEur. In other embodiments, the foams are substantially free of emulsifier.

In some embodiments, the foams comprise an emulsifier. In specific embodiments, the emulsifier is selected from the group consisting of phosphatidyl choline, polyglyceryl-3-methyl glucose distearate, PEG/dodecyl glycol copolymer, polyglyceryl-2 sesquioleate, polyglyceryl-3 diisostearate, polyglyceryl-3 polyricinoleate, sorbitan fatty acid, and combinations thereof. Emulsifiers are agents used to stabilize an emulsion by facilitating dispersion of the droplets in the non-miscible liquid component. In some embodiments of the present disclosure, an emulsifier may be added to the formulation to maintain a dispersion of water in the nonpolar liquid (or alternatively a dispersion of nonpolar liquid in water).

In one embodiment, the foams of the present disclosure comprise about 1 wt. % to about 20 wt. % of particles of the solid birch bark extract as disclosed herein, having an average particle size of less than about 50 μm, dispersed in about 80 wt. % to about 99 wt. % of one or more nonpolar liquids. In another embodiment, the foam of the present disclosure comprises about 10 wt. % of particles of the solid birch bark extract. In some other embodiments, the foam of the present disclosure is substantially free of solid birch bark extract particles having a size greater than about 50 μm.

In various embodiments of the present disclosure, the foams comprise an emulsion, wherein the interfacial surface tension of the emulsion is greater than about 4 mN/m determined with the pendant drop method.

In other various embodiments, the emulsion foams of the present disclosure have a foam index that is greater than about 2. The foam index is a measurement of the volume expansion; it is calculated by measuring the mass of a defined volume of the emulsion divided by the mass of the same volume of the foam.

The present disclosure also provides a method of making foams from a solid birch bark extract that in some embodiments may be dispersed in a nonpolar solvent to provide a clinically-advantageous oleogel comprising the steps of (a) contacting birch bark with a suitable solvent to form an extraction solution containing betulin and at least one triterpene; (b) separating the birch bark from the extraction solution; (c) cooling the extraction solution to crystallize a portion of the betulin and triterpene from the solution; (d) separating the crystallized betulin and triterpene; (e) drying the separated, crystallized betulin and triterpene to form a solid birch bark extract; (f) preparing an oleogel by dispersing a betulin-containing triterpene extract in a nonpolar liquid; (g) optionally storing the oleogel for a period of about 24 h; (h) adding an amount of water to thereby form an emulsion; and (i) placing said emulsion in a container charged with a pharmaceutically acceptable propellant. In some embodiments, the oleogel comprises from about 1 wt. % to about 30 wt. % of a triterpene extract as described herein dispersed in about 70 wt. % to about 99 wt. % of one or more nonpolar liquids. In other embodiments, the amount of water added to form an emulsion of the oleogel is in a ratio of about 1:100 to about 1:1, or in other embodiments, about 2:1 to about 1:2, with the nonpolar liquid. In still other embodiments, the pharmaceutically acceptable propellant is selected from the group consisting of carbon dioxide, nitrous oxide, propane, butane, isobutane, dimethyl ether, chlorofluorocarbons (CFCs), hydrochlorofluorocarbons (HCFCs) and hydrofluorocarbons (HFCs).

The present disclosure provides methods of treating various types of wounds in a patient in need thereof comprising topically administering an effective amount of a foam of the present disclosure to at least a portion of the wound requiring treatment. In some embodiments, the wound treated is selected from the group consisting of a bum, surgical skin lesions, superficial injuries; chronic wounds such as pressure ulcers, diabetic foot ulcers, chronic venous ulcers, artery insufficiency ulcers; aesthetic skin treatments such as ablative laser skin treatments, chemical peels, dermabrasion; wounds resulting from adverse drug reactions such as toxic epidermal necrolysis, Lyell syndrome, Stevens-Johnson syndrome or radiation dermatitis; rare skin diseases such as epidermolysis bullosa, pemphigus vulgaris or pemphigoid, and combinations thereof.

The present disclosure also provides methods of treating various diseases or conditions that result in or are associated with wounds requiring treatment. In one embodiment, a method is provided for treating epidermolysis bullosa in a patient in need thereof comprising topically administering to at least a portion of a wound resulting from or associated with epidermolysis bullosa in the patient an effective amount of a foam as presently disclosed. In another embodiment, the method is useful in treating necrotizing herpes zoster in a patient in need thereof comprising topically administering to an area of the skin undergoing necrosis in such a patient an effective amount of a foam presently disclosed.

The present disclosure provides a comparison of the wound healing effect of the betulin-containing foams of the present disclosure with those of betulin-containing oleogels, for example by comparing the betulin permeation from these novel foams to the permeation of betulin from oleogels, which have already been shown to promote wound healing. In some embodiments, the foams of the present disclosure are water-in-oil foams. The skin permeation rates of betulin, the main constituent of the foams of the present disclosure, were studied. Special emphasis was put on the influence of (1) the depth of the skin lesion of artificially injured skin and (2) the different types of oils used as a carrier.

### DETAILED DESCRIPTION OF THE DRAWINGS

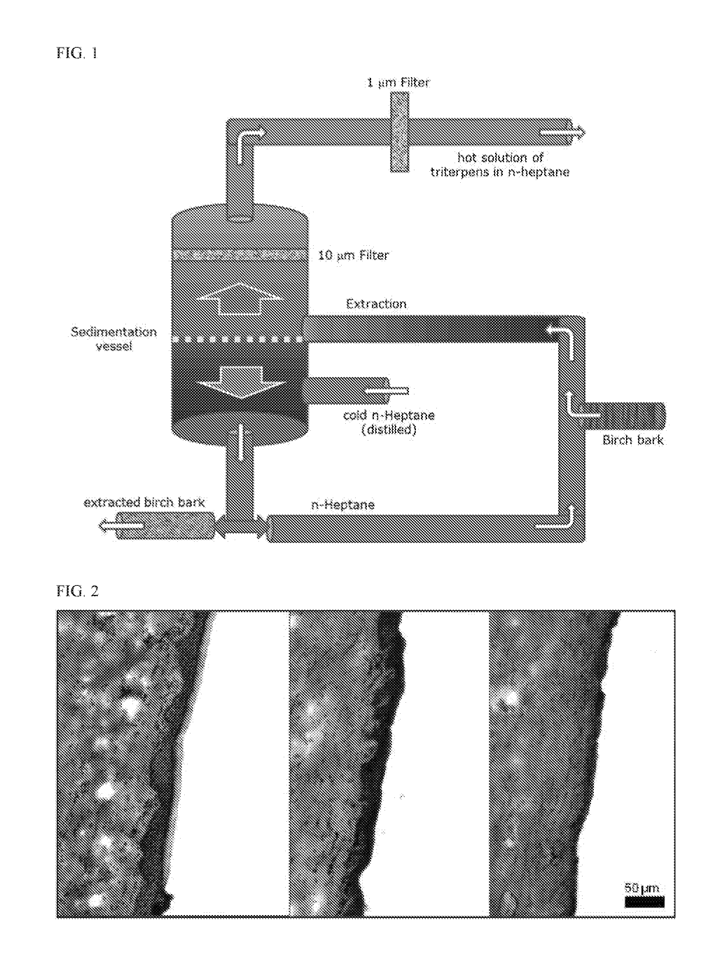

FIG. 1 illustrates the continuous extraction of birch bark to provide an extraction solution comprising betulin and one or more triterpenes.

FIG. 2 is a microscopic image of untreated porcine skin (left), skin after tape stripping (middle), and grafted skin (right).

FIG. 3 shows a comparison of betulin permeation from sunflower oil oleogels through differently injured skin and FTS; n=5; error bars: standard deviation.

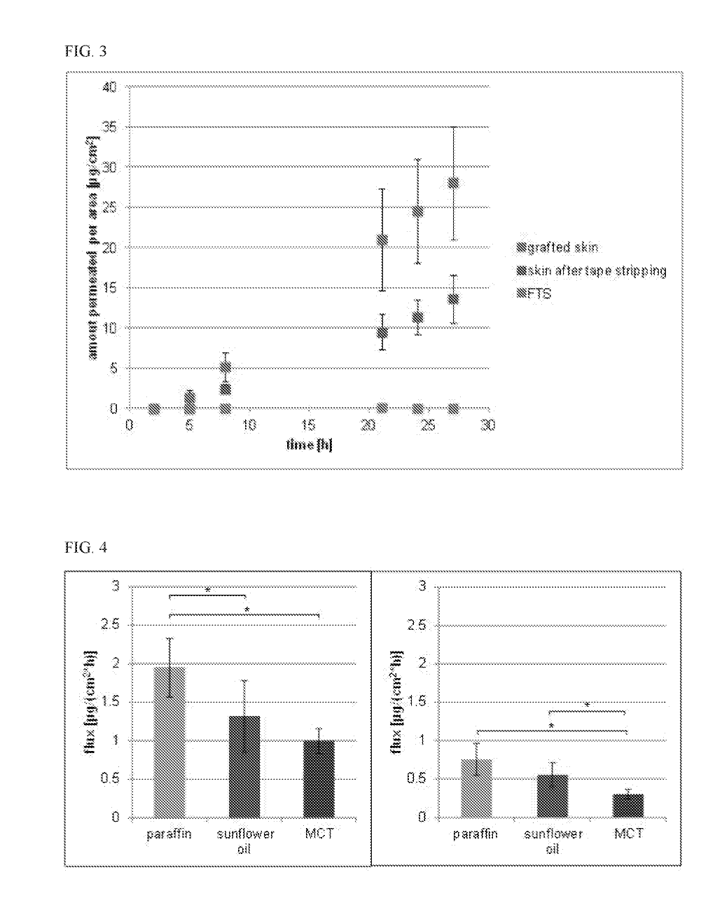

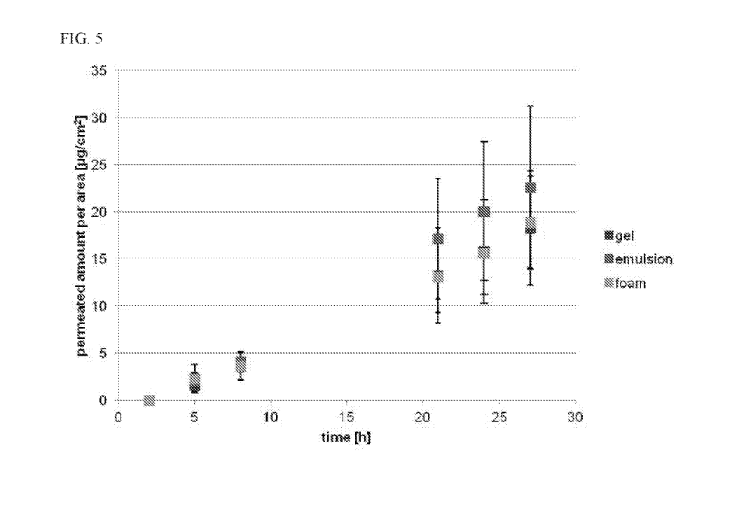

FIG. 4 shows the permeation flux of three different oleogels through grafted skin (left), and skin after tape stripping (right); n=5; error bars: standard deviation; *p<0.05.

FIG. 5 is a comparison of betulin permeation from foam, emulsion, and oleogel containing MCT through grafted skin; n=5; error bars: standard deviation.

### DETAILED DESCRIPTION OF THE DISCLOSURE

While the following terms are believed to be well understood by one of ordinary skill in the art, the following definitions are set forth to facilitate explanation of the presently disclosed subject matter.

It is to be understood that the terminology used herein is for the purpose of describing particular embodiments only and is not intended to be limiting.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein (including U.S. Pat. Nos. 9,352,041; 8,828,444; 8,536,380; and 7,482,383) are incorporated for all purposes by reference in their entirety.

Following long-standing patent law conventions, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±15% or ±10%, in another example ±5%, in another example ±1%, and in yet another example ±0.1% from the specified amount, as such variations are appropriate to perform the disclosed method.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the verb “comprise” as is used in this description and in the claims and its conjugations are used in its non-limiting sense to mean that items following the word are included, but items not specifically mentioned are not excluded.

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

“Administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound. “Administering” can also include providing directions to carry out a method involving a particular compound or a dosage form comprising the compound.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

“Therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

The term “birch bark” means the cortex of white-barked birch trees. Preferred embodiments include birch bark derived from Betula pendula Roth and Betula pubescens Ehrh as well as hybrids of both species.

The present disclosure provides solid birch bark extracts that may be formulated into clinically-advantageous emulsion foams.

The solid birch bark extracts of the present disclosure may be characterized on the basis of their chemical composition. In some embodiments, the solid birch bark extracts of the present disclosure comprise lupane and oleanane triterpenes. In particular, the birch bark extracts may contain the lupane triterpenes: betulin, lupeol, and betulinic acid, and the oleanane triterpenes: erythrodiol and oleanolic acid.

In some embodiments, the solid birch bark extract comprises at least about 50 wt. %, at least about 55 wt. %, at least about 60 wt. %, at least about 65 wt. %, at least about 70 wt. %, at least about 75 wt. %, at least about 80 wt. %, at least about 85 wt. %, or at least about 90% by weight betulin and one or more triterpenes. In some embodiments, the one or more triterpenes is selected from the group consisting of betulinic acid, oleanolic acid, erythrodiol and lupeol.

In some embodiments, the solid birch bark extract comprises at least one of the following substances: 3-b-caffeoyl betulin, acetate of the methylester of betulinic acid, acetyloleanolic acid, allobetulin, betulinic aldehyde, betulonic acid, betulonic aldehyde, lupane-3β,20,28-triol, lupane-3β,20-diol (monogynol), oleanolic aldehyde, sitosterol, ursolic acid, or β-amyrin

The solid birch bark extract of the present disclosure may be characterized by the particle size of the particles of the solid birch bark extract. In some embodiments, the average particle size of the particles of the solid birch bark extract is less than about 100 μm, less than about 90 μm, less than about 80 μm, less than about 70 μm, less than about 60 μm, less than about 50 μm, less than about 40 μm, less than about 30 μm or less than about 25 μm.

In other embodiments, the solid birch bark extract of the present disclosure is substantially free of solid birch bark extract particles having a particle size greater than about 30 μm, greater than about 40 μm, greater than about 50 μm, greater than about 60 μm, greater than about 70 μm, greater than about 80 μm, greater than about 90 μm or greater than about 100 μm.

In preferred embodiments, the solid birch bark extracts are derived from Betula pendula Roth and Betula pubescens Ehrh as well as hybrids of both species.

The present disclosure provides methods for preparing solid birch bark extracts that may be formulated into clinically-advantageous emulsion foams. In general, the methods include the steps of obtaining birch trees, stripping and processing the bark from said birch trees, contacting the processed birch bark with a suitable solvent to provide an extraction solution comprising betulin and one or more triterpenes, and isolating and drying the birch bark extract comprising betulin and one or more triterpenes from the extraction solution. In some embodiments, the isolated birch bark extract is in the form of a solid. For example, the birch bark extracts of the present disclosure can be prepared by the methods disclosed in U.S. Pat. Nos. 7,482,383, 8,536,380, 8,828,444 9,352,041, each of which is incorporated herein by reference in its entirety for all purposes.

The present disclosure provides clinically-advantageous wound-healing emulsion foam compositions and formulations comprising solid birch bark extracts that are useful as topical wound healing agents.

In various embodiments, the foams of the present disclosure comprise emulsions. In one embodiment, the emulsions useful in preparing the emulsion foams of the present disclosure are obtained from oleogels. Gels are finely dispersed systems comprising a liquid phase and a solid phase. The solid phase forms a coherent three-dimensional framework, and the two phases permeate one another. Oleogels are hydrophobic gels based on a nonpolar liquid (for example, an oil, a wax, or a paraffin) to which a gel-forming agent is added to achieve the desired physical properties.

In one embodiment, the present disclosure provides emulsion foams from oleogels comprising a nonpolar liquid and an oleogel-thrming agent. Suitable nonpolar liquids for use in oleogels of the present disclosure include, for example, plant, animal, or synthetic oils, waxes, and paraffins. In various embodiments, the nonpolar liquids are lipids. In some embodiments, the nonpolar liquid is a vegetable oil selected from the group consisting of: castor oil, peanut oil, jojoba oil, sunflower oil, olive oil, avocado oil, and almond oil. In a specific embodiment, the nonpolar liquid is sunflower oil.

In some embodiments, the nonpolar liquids are medium chain triglycerides. In some embodiments, the nonpolar liquid comprises at least one triglyceride. In certain embodiments, the at least one triglyceride is Miglyol. In other embodiments, the nonpolar liquid comprises at least one C7 or greater hydrocarbon. In certain embodiments, the at least one C7 or greater hydrocarbon is a paraffin.

In various embodiments, the peroxide value of the nonpolar liquid is less than 15, less than 14, less than 13, less than 12, less than 11, less than 10, less than 9, less than 8, less than 7, less than 6, less than 5, less than 4, less than 3, less than 2, less than 1, and including all values therebetween. In one embodiment, the present disclosure provides emulsion foams, wherein the nonpolar liquid has a peroxide value of less than about 10. In certain embodiments, the nonpolar liquid has a peroxide number of no more than about 3. The term “peroxide value” is a term known in the art to describe the extent of autoxidation the nonpolar liquid has undergone. Lower values indicate less decomposition of the nonpolar liquids.

The present disclosure provides methods of making foams from emulsions comprising oleogels. In some embodiments, after the solid birch bark extract is dried, about 1 wt. % to about 20 wt. % of the dried solid birch bark extract is dispersed in nonpolar liquid to form an oleogel. In certain embodiments, the nonpolar liquid is sunflower oil. As a general approach, the oleogel can be emulsified to form an emulsion by adding water (e.g., by a syringe-to-syringe technique, or using a high shear mixer or other large scale method) to yield a homogeneous water-in-oil emulsion, and that can be dispensed from a container as a foam using a pharmaceutically acceptable propellant.

In certain embodiments, the oleogel is sterile. The oleogel may be sterilized by suitable methods known to those skilled in the art.

In some embodiments, the oleogel prior to emulsification comprises between about 1 wt. % and about 30 wt. % solid birch bark extract (TE) dispersed in about 70 wt. % to about 99 wt. % of one or more nonpolar liquids, wherein the oleogel contains at least one oleogel forming agent in addition to the solid birch bark extract particles. In some embodiments, the oleogel comprises between about 1 wt. % and about 20 wt. % solid birch bark extract dispersed in about 80 wt. % to about 99 wt. % of one or more nonpolar liquids, wherein the oleogel contains at least one oleogel forming agent in addition to the solid birch bark extract particles.

In other embodiments, the oleogel prior to emulsification comprises between about 1 wt. % and about 30 wt. % solid birch bark extract particles dispersed in about 70 wt. % to about 99 wt. % of one or more nonpolar liquids, wherein the dispersed solid birch bark extract particles are the only oleogel forming agent in the oleogel. In some embodiments, the oleogel comprises between about 1 wt. % and about 20 wt. % solid birch bark extract particles dispersed in about 80 wt. % to about 99 wt. % of one or more nonpolar liquids, wherein the oleogel contains at least one oleogel forming agent in addition to the solid birch bark extract particles.

In certain embodiments, the oleogel prior to emulsification comprises: about 5 wt. % solid birch bark extract particle dispersed in about 95 wt. % of one or more nonpolar liquids; about 10 wt. % solid birch bark extract particle dispersed in about 90 wt. % of one or more nonpolar liquids; about 15 wt. % solid birch bark extract particles dispersed in about 85 wt. % of one or more nonpolar liquids; or about 20 wt. % solid birch bark extract particles dispersed in about 80 wt. % of one or more nonpolar liquids.

In the foregoing embodiments, the amount of solid birch bark extract particles (for example, about 1 wt. % and about 20 wt. %) includes up to about 0.5 wt. % of solid birch bark extract particles that are dissolved in the nonpolar liquid.

As described herein, the present disclosure provides methods for preparing emulsion foams comprising solid birch bark extract. The term emulsion relates to heterogeneous systems consisting of two liquids that are not miscible with each other or only miscible to a limited extent, which are typically designated as phases. In an emulsion, one of the two liquids is dispersed in the other liquid in the form of minute droplets.

In some embodiments, an emulsion comprising the solid birch bark extract of the present disclosure is provided. Other embodiments provide emulsions comprising the oleogels of the present disclosure. In various embodiments, the emulsions of the present disclosure are provided by dispersing a polar liquid in the nonpolar liquid. In specific embodiments, the polar liquid is water.

In some embodiments, the emulsions of the present disclosure include an emulsifier. In some embodiments, the emulsifier is a surfactant or other ingredient that promotes the stability of the emulsion. In certain embodiments, the emulsifier is (hydroxypropyl)methyl cellulose. In certain other embodiments, the emulsions are substantially free of an emulsifier.

For treating certain skin wounds, foams may offer several advantages over oleogels because foams can be applied to wounds almost touchless, whereas the application of an oleogel requires touch. Foams are generally based on emulsions where a propellant is mixed with the dispersed lipid phase of an emulsion. In some embodiments the propellant is carbon dioxide (CO₂). In still other embodiments, the propellant is one or more of propane, butane, isobutane, dimethyl ether, chlorofluorocarbons (CFCs), hydrochlorofluorocarbons (HCFCs), hydrofluorocarbons (HFCs), and nitrous oxide (N₂O).

The present disclosure provides foams comprising a solid birch bark extract-containing emulsion as described above.

In certain embodiments, the emulsion comprises an oleogel consisting of about 5 wt. % to about 10 wt. % solid birch bark extract, wherein the emulsion is a water-in-oil emulsion consisting of the oleogel and about 20 wt. % to about 30 wt. % of water.

In certain other embodiments, the emulsion comprises an oleogel consisting of about 7 wt. % solid birch bark extract, wherein the emulsion is a water-in-oil emulsion consisting of the oleogel and about 25 wt. % of water.

In certain embodiments, the foams of the present disclosure further comprise an emulsifier. In certain further embodiments, the emulsifier is selected from the group consisting of phosphatidyl choline, polyglyceryl-3-methyl glucose, PEG/dodecyl glycol copolymer, polyglyceryl-2 sesquioleate, polyglyceryl-3 diisostearate, polyglyceryl-3 polyricinoleate, sorbitan fatty acid esters, etc., and combinations thereof.

In certain embodiments, the foams of the present disclosure possess certain physical properties. In some embodiments, the foam index is greater than about 2. In other embodiments, the emulsion used in the foam exhibits an interfacial surface tension of greater than about 4 mN/m determined with the pendant drop method.

The present disclosure also provides for pressurized containers filled with an emulsion of the present invention and a pharmaceutically acceptable propellant whereby the emulsion forms a foam upon decanting at least a portion of the mixture from the container.

In various embodiments, the present disclosure also provides methods of treating a wound in a patient by topically administering an effective amount of a foam of the present disclosure to at least a portion of the wound.

In certain embodiments, the wound treated is selected from the group consisting of burns, surgical skin lesions, superficial injuries; chronic wounds such as pressure ulcers, diabetic foot ulcers, chronic venous ulcers, artery insufficiency ulcers; aesthetic skin treatments such as ablative laser skin treatments, chemical peels, dermabrasion; wounds resulting from adverse drug reactions such as toxic epidermal necrolysis, Lyell syndrome, Stevens-Johnson syndrome or radiation dermatitis, rare skin diseases such as epidermolysis bullosa, pemphigus vulgaris or pemphigoid, and combinations thereof.

The present disclosure also provides methods that are useful in treating various diseases and conditions afflicting a patient that result in formation of a wound comprising topically administering an effective amount of a foam described herein to an area of a wound of a patient in need thereof. In various embodiments, the diseases or conditions are selected from the group comprising burns, surgical skin lesions, superficial injuries; chronic wounds such as pressure ulcers, diabetic foot ulcers, chronic venous ulcers, artery insufficiency ulcers; aesthetic skin treatments such as ablative laser skin treatments, chemical peels, dermabrasion; wounds resulting from adverse drug reactions such as toxic epidermal necrolysis, Lyell syndrome, Stevens-Johnson syndrome or radiation dermatitis, rare skin diseases such as epidermolysis bullosa, pemphigus vulgaris, and so forth.

### EXAMPLES

### Example 1

### Preparation of the Foams

Triterpene extract from the outer bark of birch, TE, was obtained from Birken AG, Niefern-Öschelbronn, Germany, and had the composition and physical properties shown in Table 1. For all of the tested formulations, paraffin, sunflower oil or medium-chain triglycerides served as the basis of the different oleogels. These oleogels containing 10% (w/w) TE were prepared by dispersing the TE in the respective oil using an Ultra-Turrax T25 (IKA, Staufen, Germany) at 8000 rpm for 3 min. After a storage period of 24 h, the same amount of water was added to the oleogels using a syringe-to-syringe technique yielding homogenous w/o emulsions. 50 ml of the emulsions were filled in aluminum aerosol cans and charged daily with CO₂ in order to obtain a constant equilibrium pressure of 5 bar after 5 days.

TABLE 1
Chemical composition and physical characteristics of the TE used

| Chemical Composition | Specific surface area | Particle size D50% |
|---|---|---|
| 81.6% Betulin | 42 ± 0.4 m²/g | 5.8 μm |
| 3.84% Betulinic acid | | |
| 2.08%, Lupeol | | |
| 1.05% Erythrodiol | | |
| 0.97% Oleanolic acid | | |
| 0.52% Metulinic acid | | |
| methylester 9.94% | | |
| unidentified substances | | |

### Example 2

### Preparation of the Porcine Skin

The pig ears were washed with isotonic saline solution, cleaned of blood with cotton swaps and dried. The excised postauricular skin was wrapped in aluminum foil and stored at −30° C. On the day of the experiment, it was thawed at room temperature and was pinned to a styrofoam block. If not otherwise pre-treated as described below, the porcine skin was then cut with the dermatome (Dermatom GA 630, Aesculap AG & Co. KG) to a thickness of 0.8 mm.

Injuring skin: Superficial wounds are limited to the outer skin layers and are often caused by abrasion. Depending on the depth of the abrasion process the different layers of the epidermis or the dermis can be involved. The severity of the injury has a clear impact on the healing process, and also on the penetration of an active as the barrier properties of the remaining skin tissue varies.

In order to simulate two kinds of abrasion, porcine skin was “injured” for this study in two different ways. To this end, the skin was prepared as previously described and subsequently injured by either of the following two methods:
i. The first method involved skin (tape) stripping to remove the horny layer of the skin. For tape stripping, the skin on the styrofoam block was pressed to the tape (tesa No 4124, Beiersdorf AG, Hamburg, Germany) and stripped in one quick move. This procedure was repeated 20 times.
ii. The second method involved skin grafting using a dermatome to remove the outer 200 μm of the skin. For skin grafting, 0.2 mm of the outermost layers of the skin were removed by the means of a dermatome (Dermatom GA 630, Aesculap AG & Co. KG), cutting directly in the living layers of the skin.

All three kinds of skin (untreated, skin after tape stripping and grafted skin) were finally dermatomed to a constant thickness of 0.8 mm. The uniform severity of both types of injuries was confirmed through light-microscopic examination.

### Example 3

### Microscopic Examination of Injured Skin

To determine the severity of the injury, microscopic images were taken. The skin was treated as described before and frozen in liquid nitrogen. Cross-sections were cut with a cryomicrotome (HM 560 Cryo-Star; Thermo Fisher Scientific Inc.; Langenselbold, Germany). The sections had a thickness of 50 μm and were stained with hematoxylin and eosin before taking microscopic images (Microscope Axio Imager Z1, Carl Zeiss, Jena, Germany).

The microscopic images in FIG. 2 show the severity of the damage to the skin after the two different treatments, tape stripping and skin grafting, compared to the untreated, full thickness skin (FTS). The untreated skin shows the typical layer structure of skin including stratum corneum, epidermis and dermis. After tape stripping, the stratum corneum as the outermost layer of the skin has been removed completely from the skin. In contrast, skin grafting leads to a more severe damage, cutting directly deep into the living epidermal layers of the skin.

### Example 4

### Permeation Experiments

Betulin Assay:

Betulin was quantified by HPLC using the following system: LC-20A prominence HPLC system (Shimadzu, Duisburg, Germany), HPLC column Nucleosil 100-5 C18 EC 125/4, HPLC pre-column Universal RP EC 4/3 (both Macherey-Nagel, Düren, Germany). The temperature for the column was set to 40° C. and the flow rate to 1.5 mL/min. The composition of the mobile phase was 70% of acetonitrile and 30% of water. Limit of detection was 0.0491 μg/ml and limit of quantification 0.1473 μg/ml. A volume of 20 μl of every sample was injected and the UV absorbance was measured at 210 nm. The retention time for betulin was approx. 10.3 min.

Statistical Analysis:

All data was obtained by repeating the measurements (n≥4), and analyzed by one-way (single factor) analysis of variance followed by the Student-Newman-Keuls test.

Ex Vivo Permeation Experiments:

Permeation experiments were performed using modified Franz-type diffusion cells (Gauer Glas, Püttlingen, Germany) with a receptor-volume of 12 ml. Phosphate buffered saline pH 7.4 was used as receptor fluid with 10% hydroxypropyl-β-cyclodextrin to enhance the solubility of betulin. The receptor fluid was preheated to 32° C. and filled into the diffusion cells. Skin samples (thickness 0.8 mm, diameter 2.5 cm) were obtained from porcine skin that was either untreated retaining the natural skin barrier or “injured” by either tape stripping or by skin grafting. The donor compartment was fitted to the cells and they were heated to 32° C. in a water bath followed by an equilibrium time of 30 min. For infinite dose experiments, 1 g of the formulations was applied uniformly on the porcine skin. The diffusion cells were capped with Parafilm® to avoid water evaporation. The stirring speed of the receptor fluid was 500 rpm. Samples of 0.5 ml were taken after 2 h, 5 h, 8 h, 21 h, 24 h and 27 h and replaced by fresh preheated receptor fluid. To obtain the amount of permeated betulin, the samples were analyzed via HPLC. The cumulative permeated amount of betulin per area was plotted against the time. The flux of the permeation was calculated using the results from 8 h to 27 h. The first two samples were excluded for linear regression as the flux was not yet in a steady state in all the experiments. All experiments were performed in quintuplicate.

Permeation Through Differently Injured Skin:

First, permeation of betulin from sunflower oleogels through the different types of skin was investigated. Intact skin displays an almost perfect barrier function, and prevents the delivery of xenobiotic molecules like betulin across the skin. Experiments with FTS revealed a marginal permeation of betulin with a value below the limit of quantification (0.88 μg/cm2 FIG. 3). As expected, permeation flux was significantly higher when the barrier function of the skin was artificially impaired. As tape stripping only removes the stratum corneum, the flux after tape stripping (0.44±0.11 μg/cm2*h) was only half the value than through grafted skin (1.13±0.15 μg/cm2*h). Furthermore, the lag time, defining the time taken until betulin initially enters the receptor fluid, is shorter for the permeation through the skin damaged by a dermatome compared to stripped skin (4.02±1.02 vs. 6.51±1.48 h). As the flux is inversely proportional to the thickness of the skin, this variable was kept constant throughout all experiments. Therefore it is only influenced by the diffusion coefficient which is dependent on the structure of tissue that has to be crossed. Although after the injury of the skin by tape stripping and grafting the stratum corneum as the major barrier has been removed there is still a different resistance to betulin permeation. A possible explanation might be that after tape stripping a smooth surface remains whereas grafting cuts directly into the intact tissue and opens additional pathways for the permeating triterpenes. In the context of wound healing this means that the deeper the injury, the more betulin penetrates the skin per unit time, and a more pronounced wound healing effect can be expected with increasing severity of a wound.

### Example 5

### Comparison of Permeation from Oleogels Comprising Different Oils:

Solubility and gel strength of TE oleogels depend strongly on the polarity of the lipid used, but show no simple correlation due to a complex overlapping of several effects. In order to evaluate if the nature of the used oil has an impact on betulin permeation we choose for this study sunflower oil which is of medium polarity and has already proven to enhance on wound healing. As second triglyceride with similar polarity but higher solubility, MCT was selected. Paraffin was selected as an example of a nonpolar lipid. The properties of the selected oils are summarized in Table 2. Interestingly, permeation flux of the oleogels prepared with different oils showed a clear trend regarding the betulin flux which was independent of the severity of the injury. For grafted skin as well as for skin after tape stripping, the flux increases in dependence from the used lipid phase in the following order: MCT<sunflower oil<paraffin (FIG. 4). All oleogels share the fact that the oil is saturated with an excessive amount of TE suspended as solid particles in the oil phase. Consequently, the oil is saturated with betulin and its activity coefficient in all oleogels is 1. In all cases the concentration in the skin should yield its saturation concentration when distribution equilibrium is achieved. However, skin permeation is a dynamic process and might also depend on the release kinetic of the active from the vehicle. Obviously, the different viscosities of the oleogels affect the transport of the active to the skin. The respective apparent viscosities are summarized in Table 3. Comparing flux and viscosity shows that there is a clear relationship but not a full correlation as rheological measurements characterize the macro-viscosity of a system whereas diffusion and release are influenced by the micro-viscosity. Interestingly the observed order in the permeation flux with the different oils differs from the release rate measured from equivalent oleogels. This indicates that although the stratum corneum as the predominant skin barrier has been removed for these experiments there is a specific interaction between the oil which is used as vehicle and the injured skin.

TABLE 2
Interfacial tension, solubility, and log P values of TE with different oils

| | interfacial tension [mN/m] | TE solubility [mg/mL] | log P |
|---|---|---|---|
| paraffin | 42.7 | 0.41 | 3.21 |
| sunflower oil | 25.4 | 4.4 | 4.24 |
| MCT | 27.1 | 7.9 | 4.50 |

TABLE 3
Viscosities of the different oleogels; n = 3; mean ± standard deviation

| oil phase | viscosity [Pas] |
|---|---|
| paraffin | 0.11 ± 0.025 |
| sunflower oil | 0.41 ± 0.028 |
| MCT | 0.48 ± 0.026 |

### Example 6

### Comparison of Permeation from Different Formulations Containing the Same Oil

Finally, the different formulation types (oleogel, emulsion, and foam) based on the same oil were examined. As can be seen in FIG. 5 exemplary for formulations containing MCT and in Table 4, all types of formulations revealed almost identical permeation kinetics for betulin when applied to skin with the same pretreatment. This can be attributed to the fact that the oleogel with an excess amount of TE forms the outer phase of all types of formulation and therefore is likewise in direct contact to the skin. As a result, neither water, nor CO₂ which when present are located in the inner phase of these formulations significantly affect permeation flux or lag time. Note: The permeation values of oleogel, emulsion, and foam are to be compared for the same oil and the same injury only.

In conclusion, this study showed that foams prepared from emulsions with TE as an active ingredient lead to permeation rates through injured skin which are comparable to the corresponding oleogels, which have proven to promote wound healing. This result is surprising because an emulsion foam would be expected to provide a lower topical dose of active agent compared to an oleogel, as the emulsion foam includes a significant volume of active ingredient-free polar liquid (e.g., water), and void volume (e.g., voids produced by the foaming agent/propellant) compared to the oleogel. This would be expected to reduce the level of permeation of the active ingredient into the wound site. Thus, unexpectedly, the foams provide an advantageous application form in wound treatment, which combines the positive effects of the birch bark dry extract with the advantages of the application form that allows virtually touchless application.

TABLE 4
Comparison of flux and lag time of betulin from the different
formulations; n = 4-5; mean ± standard deviation

| | | | flux | |
|---|---|---|---|---|
| used oil | kind of injury | formulation | [μg/cm² * h] | lag time [h] |
| sunflower oil | tape stripping | gel | 0.44 ± 0.11 | 6.51 ± 1.48 |
| | | emulsion | 0.36 ± 0.11 | 5.70 ± 2.18 |
| | | foam | 0.43 ± 0.14 | 4.34 ± 2.24 |
| | grafting | gel | 1.13 ± 0.15 | 4.02 ± 1.02 |
| | | emulsion | 1.15 ± 0.13 | 3.37 ± 0.64 |
| | | foam | 1.29 ± 0.25 | 3.32 ± 1.15 |
| paraffin | tape stripping | gel | 1.02 ± 0.17 | 4.01 ± 2.07 |
| | | emulsion | 0.87 ± 0.42 | 3.21 ± 3.11 |
| | | foam | 0.98 ± 0.31 | 2.46 ± 2.29 |
| | grafting | gel | 1.95 ± 0.39 | 4.06 ± 1.29 |
| | | emulsion | 1.67 ± 0.45 | 4.82 ± 1.28 |
| | | foam | 1.45 ± 0.50 | 5.02 ± 1.88 |
| MCT | tape stripping | gel | 0.58 ± 0.17 | 7.47 ± 0.79 |
| | | emulsion | 0.76 ± 0.22 | 6.38 ± 1.97 |
| | | foam | 0.65 ± 0.22 | 6.49 ± 1.07 |
| | grafting | gel | 0.76 ± 0.34 | 2.72 ± 2.03 |
| | | emulsion | 0.98 ± 0.47 | 3.51 ± 2.05 |
| | | foam | 0.78 ± 0.20 | 3.69 ± 1.72 |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. An emulsion foam comprising solid birch bark extracts dispersed in one or more nonpolar liquids.

2. The foam of claim 1, wherein the birch bark extracts comprise at least about 70 wt. % betulin and one or more triterpenes selected from the group consisting of betulinic acid, oleanolic acid, erythrodiol, and lupeol.

3. The foam of claim 1, wherein the nonpolar liquid is selected from the group consisting of sunflower oil, medium chain triglycerides, and paraffin.

4. The foam of claim 1, further comprising water.

5. The foam of claim 1, wherein the emulsion is a water-in-oil emulsion.

6. The foam of claim 5, wherein the emulsion comprises an oleogel.

7. The foam of claim 6, wherein the oleogel consists of about 5 wt. % to about 10 wt. % solid birch bark extract and the emulsion is a water-in-oil emulsion consisting of the oleogel and about 20 wt. % to about 30 wt. % of water.

8. The foam of claim 7, wherein the oleogel consists of about 7 wt. % solid birch bark extract and the amount of water in the emulsion is about 25 wt. %.

9. The foam of claim 1 comprising about 1 wt. % to about 20 wt. % of particles of the solid birch bark extract of any one of claims 1-10, having an average particle size of less than about 50 dispersed in about 80 wt. % to about 99 wt. % of one or more nonpolar liquids.

10. The foam of claim 9, comprising about 10 wt. % of particles of the solid birch bark extract.

11. The foam of claim 1, wherein the nonpolar liquid comprises at least one triglyceride.

12. The foam of claim 1, wherein the nonpolar liquid comprises at least one C7 or greater hydrocarbon.

13. The foam of claim 12, wherein the nonpolar liquid comprises one or more vegetable oils.

14. The foam of claim 13, wherein the nonpolar liquid comprises sunflower oil.

15. The foam of claim 11, wherein the triglyceride is medium chain triglycerides

16. The foam of claim 1, wherein the nonpolar liquid has a peroxide value less than about 10.

17. The foam of claim 16, wherein the peroxide value is no more than about 3.

18. The foam of claim 1, wherein the foam is substantially free of solid birch bark extract particles having a size greater than about 50 μm.

19. The foam of claim 1, wherein the foam is sterile.

20. The foam of claim 1, substantially free of emulsifier.

21. The foam of claim 1, wherein the interfacial surface tension of the emulsion is greater than about 4 mN/m (pendant drop method).

22. The foam of claim 1, wherein the foam index is greater than about 2.

23. The foam of claim 1, further comprising an emulsifier selected from the group consisting of phosphatidyl choline, polyglyceryl-3-methyl glucose distearate, PEG/dodecyl glycol copolymers, polyglyceryl-2 sesquioleate, polyglyceryl-3 diisostearate, polyglyceryl-3 polyricinoleate, sorbitan fatty acid esters, and combinations thereof.

24. The foam of claim 1, prepared by a process comprising:
(a) preparing an oleogel by dispersing betulin-containing triterpene extracts in a nonpolar liquid;
(b) optionally storing the oleogel for a period of about 24 h;
(c) adding an amount of water by a method that forms an emulsion; and
(d) placing said emulsion in a container charged with a pharmaceutically acceptable propellant.

25. The foam of claim 24, wherein the oleogel of step (a) comprises from about 1 wt. % and about 30 wt. % triterpene extracts dispersed in about 70 wt. % to about 99 wt. % of one or more nonpolar liquids.

26. The foam of claim 24, wherein the amount of water added in step (c) is in a ratio of about 1:100 to about 1:1 with the nonpolar liquid.

27. The foam of claim 24, wherein the pharmaceutically acceptable propellant of step (d) is selected from the group consisting of carbon dioxide, nitrous oxide, propane, butane, isobutane, dimethyl ether, one or more chlorofluorocarbons (CFCs), one or more hydrochlorofluorocarbons (HCFCs) and one or more hydrofluorocarbons (HFCs).

28. A method of treating a wound in a patient comprising topically administering an effective amount of the foam of claim 1 to at least a portion of the wound.

29. The method of claim 28, wherein the wound treated is selected from the group consisting of a burn, surgical skin lesions, superficial injuries; chronic wounds, pressure ulcers, diabetic foot ulcers, chronic venous ulcers, artery insufficiency ulcers; aesthetic skin treatments, ablative laser skin treatments, chemical peels, dermabrasion, wounds resulting from adverse drug reactions, toxic epidermal necrolysis, Lyell syndrome, Stevens-Johnson syndrome, radiation dermatitis, rare skin diseases, epidermolysis bullosa, pemphigus vulgaris and pemphigoid.

30. A method of treating epidermolysis bullosa in a patient in need thereof comprising topically administering to an area of epidermolysis bullosa of the patient an effective amount of the foam of claim 1.

31. A method of treating necrotizing herpes zoster in a patient in need thereof comprising topically administering to an area of the skin undergoing necrosis of the patient an effective amount of the foam of claim 1.

32. A pressurized container for dispensing a foam of claim 1, comprising an emulsion and a pharmaceutically acceptable propellant, whereby the emulsion forms a foam upon releasing at least a portion of the mixture from the container.
